(19) United States
(12) Patent Application Publication
(10) Pub. No.: US 2003/0003113 A1
(43) Pub. Date: Jan. 2, 2003

(54) Individualized addiction cessation therapy

(76) Inventor: Leon J. Lewandowski, West Hampton, NY (US)

Correspondence Address: CRAIG G. COCHENOUR, BUCHANAN INGERSOLL, P.C., ONE OXFORD CENTRE, 20th FLOOR, 301 GRANT STREET, PITTSBURGH, PA, 15219, US

(21) Appl. No.: 10/103,904

(22) Filed: Mar. 22, 2002

Related U.S. Application Data
(60) Provisional application No. 60/302,013, filed on Jun. 29, 2001.

(51) Int. Cl. G06F 17/60; A61K 9/00

(52) U.S. Cl. 424/400; 705/3

(57) ABSTRACT

The present invention provides pharmacologically active compositions of drugs of addiction, or their respective agonists or antagonists in a variety of unit-dose or multidose drug delivery systems, including those for transdermal, intranasal and sublingual administration, and methods of use thereof. A patient individualized addiction cessation therapy treatment method is also provided that step-wise decreases the addictive substance from the patient's central nervous system over time. A computerized data processing system and method for assisting medical practitioners in selecting a medical treatment for a patient based upon known medical and clinical data and outcomes are disclosed.

### BENEFIT OF PRIOR PROVISIONAL APPLICATION

This utility patent application claims the benefit of copending prior U.S. Provisional Patent Application Serial No. 60/302,013, filed Jun. 29, 2001, entitled “Individualized Addiction Cessation Therapy” having the same named applicant as inventor, namely Leon J. Lewandowski, as the present utility patent application.

### BACKGROUND OF THE INVENTION

1. Field of the Invention

The present invention relates to pharmacologically-active formulations of drugs of addition, or their respective agonists or antagonists, and their respective delivery systems administered by a variety of delivery routes and devices, to humans according to an individualized algorithm (treatment plan) in a controlled-manner designed to eliminate the addictive substance from the recipients central nervous system (CNS) slowly over time, allowing the subject to be free of uncontrolled cravings, thereby normalizing the brain receptor chemistry back to a pre-addiction status. During and following this critical first step of medical detoxification, psychotherapeutic counseling must be initialized and maintained, and individualized support systems (familial/workplace/societal) must be established.

The variety of drugs of addiction include, but are not limited to, those in such categories as, (a) opioids and morphine-derivatives, (b) stimulants, (c) depressants, (d) cannabinoids, (e) dissociative anesthetics, and (f) hallucinogens, etc. The variety of delivery routes include, but are not limited to, (a) intranasal, to the nasal mucosa (b) transdermal (patch), to the skin (c) sublingual, to the oral mucosa below the tongue (d) oral, to the mouth for swallowing (e) inhalation, to the mucosa of the respiratory tract (f) intravenous, by injection to the blood (g) subdermal/intramuscular (deposition), by injection.

The variety of devices for delivery of a wide range of therapeutically-active concentrations of drugs of addiction, or their respective agonists or antagonists include, such as for example, but are not limited to, (a) squeeze-activated unit-dose and multiple-dose mechanical-pump dispensers for intranasal delivery of aqueous mist, gel droplet, or powder (b) skin-patch therapeutic systems for controlled transdermal delivery (c) quick-dissolve tablets for sublingual delivery (d) controlled (timed) release solid form standard tablets and capsules/caplets for gastrointestinal delivery (e) aqueous mist solutions or powders for inhalation/respiratory tract delivery (f) pre-filled syringes with accident-proof needles for injection delivery

2. Brief Description of the Background Art

The effects of most drugs result from their interaction with macromolecular components of the organism. Such interaction alters the function of the pertinent target component and thereby initiates the biochemical and physiological changes that are characteristic of the drug. The terms “receptive substance” and, more simply, “receptor” were coined to denote the target component of the organism with which the chemical agent was presumed to interact. In general terms, many drugs act on such physiological receptors. Those drugs that mimic to any degree (wholly or partially) the effect of the initial regulatory compounds (in this specific case, opioid analgesics) are termed “agonists”, those that also bind to the target receptor but have no intrinsic mimicking regulatory activity, but thereby interfere with the effects of the initial regulatory compounds, are termed “antagonists”.

The subject of “drug addiction” is multifaceted and many aspects of it (even some of the definitions) are controversial. All addictive drugs mimic (or occasionally block) the actions of some neurochemical transmitter. Progress in the understanding of the causes of addictions and its treatment has been impeded by the lack of a unifying biochemical theory. However, recent evidence suggests that some common mechanism might underlie addictions to otherwise apparently unrelated drugs. A major hypothesis has emerged suggesting that the neurotransmitter dopamine (DA) might play a central role in the molecular mechanisms of at least some addictions, including not just to drugs, but also possibly to sex, gambling and even overeating of foods. (Addiction to other drugs are thought to involve other universal brain chemicals, such as serotonin). If so, such neurotransmitters would represent an important target for discovery of effective pharmacotherapy and revolutionize the pharmacist's role in treating addictions.

The National Institute of Drug Abuse (NIDA), lists the most “commonly abused drugs” in six major categories: opioids and morphine-derivatives, stimulants, depressants, cannabinoids, dissociative anesthetics, and hallucinogens. For example, opioids and morphine derivatives (opiate analgesics) include, such as for example, but are not limited to morphine, codeine, fentanyl, heroin, and opium. Opiate agonists, as exemplified by (but not limited to) methadone hydrochloride or its long-acting alternative levomethadyl acetate (LAMM) hydrochloride, or opiate antagonists, as exemplified by (but not limited to) naloxone or naltrexone, have been used for maintenance (long-term) treatment of, and detoxification (short-term) from opiate dependence.

Currently methadone is the only opiate agonist approved by the USFDA for detoxification treatment of opiate dependence; however, methadone and LAMM are both approved for maintenance treatment of opioid dependence.

Typically, as in the case of the key opiate agonists (methadone and LAMM) the drug is delivered orally for gastro-intestinal (GI) tract absorption, or by injection, or subcutaneously. Dispersible tablets of the drug contain insoluble excipients and are intended for dispersion in a liquid prior to oral administration (and not for injection).

Methadone treatment has been the principal approach to successful “maintenance” pharmacotherapy of opiate dependence for over 30 years. Its positive aspects include oral-dosing, a long biological half-life in humans, minimal side effects profile, relative inexpensiveness, and reasonable “success.” Methadone maintenance treatment prevents drug cravings, withdrawal symptoms, blocks euphorogenic effects of other opiates, and prevents relapse to illicit use of opiates. It does so essentially by occupying, and thereby blocking, narcotic receptors. It is postulated that the high rate of relapse after detoxification from heroin use is due to a persistent “derangement” of the narcotic receptor system, and that daily methadone maintenance compensates for this defect.

The major negative of methadone maintenance is that it substitutes one addiction for another, with perhaps an even more-addictive compound. The treatment is corrective but not curative for severe addicts. Methadone maintenance usually implies life-long treatment. Moreover, success is highly dependent on selection of highly motivated patients. While methadone maintenance programs do help society by eliminating many of the ancillary problems associated with opiate dependence, such as crime, these programs do not generally allow the addict ever to return to a drug free existence.

To improve on any disadvantages associated with the current oral and parenteral administration of methadone/LAMM, in particular its own highly-addictive propensity, there is a need in the art for pharmaceutical formulations containing methadone or LAMM that (1) provide controlled-release of drugs for a more constant CNS receptor binding with a reduced but “maintained” dose so as to reduce “bolus” systemic delivery when compared to oral or parenteral administration (thereby reducing any potential side-effect profile), and (2) provide a shorter onset of CNS receptor binding with a markedly reduced dose and a relatively rapid decrease in plasma levels which minimizes systemic delivery when compared to oral or parenteral administration (thereby minimizing any potential side-effect profile).

It is therefore, an object of the present invention to provide pharmaceutical formulations of drugs of addiction, or their agonists or antagonists, as exemplified by the opiate agonists methadone or LAMM, that provide activity of the drugs (compared to oral or parenteral administration) at a reduced dose and/or a controlled-release dose both of which reduces or minimizes systemic delivery compared to standard oral delivery.

Dopamine is a brain chemical that regulates a number of body functions, such as movement, attention motivation and pleasure. Use of drugs of abuse, over time causes an alteration in the individual's metabolism of dopamine, thereby altering available dopomine levels. Any severe alteration in dopamine release results in the withdrawal state (often described as like experiencing the flu) causing cravings which then provoke re-use of drugs so as to “correct” the deficit. This cycle is, in essence, drug-addiction.

Research shows that chronic administration of drugs results in the development of (A) “tolerance” to the pharmacological effects of the drug, and (B) physical dependence, generally associated with qualitative and quantitative changes in specific drug receptors in the central nervous system.

Addiction to any chemical substance, whether to drugs, alcohol, tobacco, etc., has certain physiological and psychological characteristics, i.e., the sudden deprivation of the addicting agent creates “withdrawal-symptoms.” Accordingly, for any treatment program to be successful it must minimize (or better yet eliminate) putting the addict through experiencing the withdrawal state (with its strong sense of deprivation and pain, both physical and emotional), thereby leading to relapse.

Programs currently in existence to deal with substance addiction generally share certain properties. (1) They have a relatively high failure rate; (2) they often entail large expenses, thereby limiting the number of people who can afford them; and (3) they do not properly address the pain and suffering created by too-sudden withdrawal from the addictive substance. Moreover, it is very difficult for one whose mind and body is racked with the discomfort of withdrawal to focus on learning new perspectives about their body, their personality, and their behavior, particularly as the latter relates to their past ways of responding to stress.

For any successful approach to the drug problem, existing programs must be expanded to approach the addict (patient) from three combined perspectives: medical, psychological and societal (social). Drug-addiction is principally a medical problem, albeit one with both psychological and social implications. Medical problems require medical treatments, first and foremost. Any program which ignores any of these three components (especially the medical component) will result in treatment failures. For example, common programs which focus only on “cold-turkey” withdrawal followed by “buddy-system” type psychological support programs probably represent the main reason why the majority of attempts to end a chemical addition end in relapse. These “talk-support” programs can be valuable but only when used following the medical/pharmacological first step of controlled detoxification under medical supervision.

Those who are wary of looking “soft on crime” but agree that the simple criminalization of the medical problem of drug addiction is not working, should remember that medical treatment is far from an “easy option” for addicts. In particular, successful detoxification treatment is often followed by rigorous, demanding and long-term life-changing processes of personal growth. The overall process requires not just abstinence and psychotherapeutic counseling, but also the development and maintenance of family, work-place, and societal support systems.

Overcoming addiction to any chemical substance requires two key consecutive goals; both of which require that the addict be “highly-motivated”: (1) Carefully-controlled elimination of the addictive substance from the subject's system; and (2) Behavior modification in which the addict recognizes both their own responsibility and accountability for surrendering voluntary control, and their need to discipline themselves to regain that control. Any psychotherapeutic counseling program must be able to provide to the patient new approaches to handling their problems of daily living and any stresses which may have contributed to the drug abuse state in the first place.

In spite of this background art, there remains a very real and substantial need for pharmacologically active formulations of drugs of addiction, or their respective agonists or antagonists, for delivery in decreasing concentrations over time, by a variety of multidose or unit-dose delivery systems, to promote addiction cessation, and methods of using the same, in transdermal delivery (patch) systems, intranasal delivery systems, and in fast-dissolve sublingual delivery systems. Further, there is a need for a computer database system and method for collecting information regarding a patents medical addiction and an algorithm for use in a process that shall assist health care providers in making informed and qualitative decisions with regard to administering proper medical treatment.

### SUMMARY OF THE INVENTION

The present invention has met the above-described needs. The present invention provides a method for reducing the exposure to the addictive agent over a period of about a day to months, depending on the individual involved. This can be accomplished with a variety of chemical substances (prescription medications, i.e., the drugs of addiction themselves, or their respective agonists or antagonists), delivered by a variety of drug delivery systems, over a variable period of time, targeted to gradually controlling any potential withdrawal symptoms and cravings, thereby minimizing the risk of relapse. The present invention recognizes that the “one-size-fits-all” formula does not work in the treatment of drug-addiction. An overall plan of treatment must be designed upfront, but any such treatment plan must be able to be individualized to the needs of each patient, as the program progresses. The length of the actual medical/pharmacological detoxification process may range from 1 to 365 days and preferably averages from four to twelve weeks. The goal is to slowly normalize the brain receptor-chemistry status of the addict back to a pre-addiction state.

The method of the present invention further includes that during and following successful medical/pharmacological detoxification, the psychotherapeutic counseling and support components of a successful treatment plan come into play. The psychotherapeutic counseling must involve medical personnel trained in “talk therapy” and monitoring the subject's health and behavior. Often anxiety and depression are secondary sequellae of drug treatment; these may, for example, also require medication. The length of the active psychotherapeutic counseling phase preferably averages four to twelve weeks, or longer if occasional “booster” counseling is needed. The length of the “societal” support-system phase is best listed as “long-term” (all depending on the individual's motivation and availability of family workplace assistance and patience).

In an embodiment of this invention, a pharmaceutical dosage unit for promoting detoxification (addiction cessation) in a mammal by transdermal administration of pharmaceutically-active amounts of drugs and exemplified by, but not limited to, the drug agonists methadone/LAMM is provided or drug antagonists such as naloxone/naltrexone. Such products would be delivered to the skin transdermally, by transdermal pharmaceutical vehicle technology known by those skilled in the art, such as for example but not limited to, in water-soluble buffered, gel compositions, preferably at a neutral pH 7.0 and with an anionic surfactant to enhance rapid absorption. The surfactant amount can be as low as 0.1 wt. %, but is not to exceed 1.0 wt. %. The surfactant is for example, but not limited to, a salt of a long chain hydrocarbon with a functional group that is, for example, but not limited to, carboxylates, sulfonates and sulfates.

The present invention also includes a method of using the above-described dosage unit to promote detoxification (addiction cessation) in a mammal. This is accomplished by administering to the skin of the mammal by employing a transdermal pharmaceutical vehicle, preferably having a neutral pH of about 7.0, having a pharmaceutically-active amount of a drug of addition, or its agonist, or its antagonist such as described herein. The dosage units of the present invention provides constant blood plasma levels of drugs after being administered to the skin of the mammal. Through the use of the dosage units of the present invention, essentially constant plasma concentrations of drugs, as exemplified by methadone/LAMM, can be maintained following administration of a transdermal delivery system, such as for example, a transdermal patch applied to the skin. Such constant (“steady -state”) delivery advantageously facilitates sufficient plasma levels in the mammal to suppress withdrawal symptoms and minimize cravings.

In another embodiment of the present invention, a pharmaceutical dosage unit for promoting detoxification (addiction cessation) in a mammal by intranasal administration of pharmaceutically-active amounts of drugs and exemplified by, but not limited to, the drug agonists methadone/LAMM or drug antagonists such as naloxone/naltrexone, is provided. Such products would be delivered to nasal mucosa in an acceptable intranasal pharmaceutical vehicle, as known by those persons skilled in the art such as for example, in aqueous, gel or powdered forms, preferably at a pH of about 7.0 and with a surfactant to enhance rapid absorption and utilizing unit-dose or multidose delivery systems. For example, preferably the surfactant is an anionic surfactant. The anionic surfactant amount is as low as 0.1 wt. %, but is not to exceed 1.0 wt. %. The anionic surfactant can be a salt of a long chain hydrocarbon with a functional group that can include, but is not limited to, carboxylates, sulfonates and sulfate. Salts of long chain hydrocarbons with sulfate functional groups are preferred with sodium lauryl sulfate being more preferred.

The present invention also includes a method of using the intranasal dosage unit to promote detoxification (addiction cessation) in a mammal. This is accomplished by administering to the nasal mucosa of the mammal a dosage unit containing the intranasal pharmaceutical vehicle, such as an aqueous buffered solution, or gel, or powder preferably having a pH of about 7.0, and a pharmaceutically-active amount of a drug addiction or its agonist, or its antagonist as described herein. Advantageously, the dosage units of the present invention provide a rapid onset of transiently increased blood plasma levels of drugs such as methadone/LAMM after being administered to the nasal mucosa of the mammal. Through the use of the dosage units of the present invention, transiently increased peak plasma concentrations of methadone/LAMM can be achieved within minutes of administration, preferably within ten minutes of administration. In addition, the dosage units of the present invention upon administration to the nasal mucosa exhibit a relatively-rapid decrease in blood plasma levels of drug after reaching a transiently increased peak plasma concentration. This advantageously facilitates a decrease in plasma levels back to “steady-state” levels in the mammal after suppressing cravings.

Another embodiment of the present invention provides a pharmaceutical dosage unit for promoting detoxification (addiction cessation) in a mammal by sublingual administration of pharmaceutically-active amounts of drugs and exemplified by, but not limited to, the drug agonists methadone/LAMM or drug antagonists such as naloxone/naltrexone. Such products would be delivered to the oral mucosa below the tongue in fast-dissolve form, preferably at a pH of about 7.0 and with an anionic surfactant to enhance rapid absorption. The amount of anionic surfactant is as low 0.1 wt. %, but is not to exceed 1.0 wt. %. The anionic surfactant can be a salt of a long chain hydrocarbon with a functional group that can include, but not limited to, carboxylates, sulfonates and sulfates. Salts of long chain hydrocarbons with sulfate functional groups are preferred with sodium lauryl sulfate being more preferred.

In yet another embodiment of the present invention, a method of using the sublingual mucosal dosage unit to promote detoxification (addiction cessation) in a mammal is provided. This is accomplished by administering to the oral mucosa below the tongue of the mammal a fast-dissolve dosage unit containing a pharmaceutical vehicle capable of being administered to effect dissolution upon the mammal's sublingual mucosa, and may include for example, a buffered formulation preferably having a pH of about 7.0, and a pharmaceutically-active amount of a drug addiction, or its agonist, or its antagonist as described herein. Advantageously, the dosage units of the present invention provide a rapid onset of transiently increased peak blood plasma levels of drugs such as methadone/LAMM after being administered to the oral mucosa below the tongue of the mammal. Through the use of the dosage units of the present invention, transiently increased plasma concentrations of methadone/LAMM can be achieved within minutes of administration, preferably within ten minutes of administration. In addition, the dosage units of the present invention, upon administration to the oral mucosa below the tongue, exhibit a relatively-rapid decrease in blood plasma levels of drug after reaching a transiently increased peak plasma concentration. This advantageously facilitates a decrease in plasma levels, back to “steady-state” levels in the mammal after suppressing cravings.

In another embodiment of the present invention, a computerized data processing system is provided for the collection of medical information and clinical results for use in assisting medical providers in making informed and qualitative decisions with regards to administering the proper medical treatments. The pharmacologically active formulations, methods of using the same and the computer database system and method of the present invention will be more fully understood from the following descriptions of the invention, the drawings and the claims appended thereto.

### BRIEF DESCRIPTION OF THE DRAWINGS

FIG. 1 is a flow diagram of the individualized addiction cessation therapy algorithm of the present invention.

FIG. 2 is a diagram of key elements of the individualized addiction cessation therapy (I-ACT) detoxification algorithm of the present invention.

FIG. 3 shows the steps of the individualized addiction cessation therapy at various drug levels over time for each of the drug delivery systems of the present invention.

### DETAILED DESCRIPTION OF THE PREFERRED EMBODIMENTS

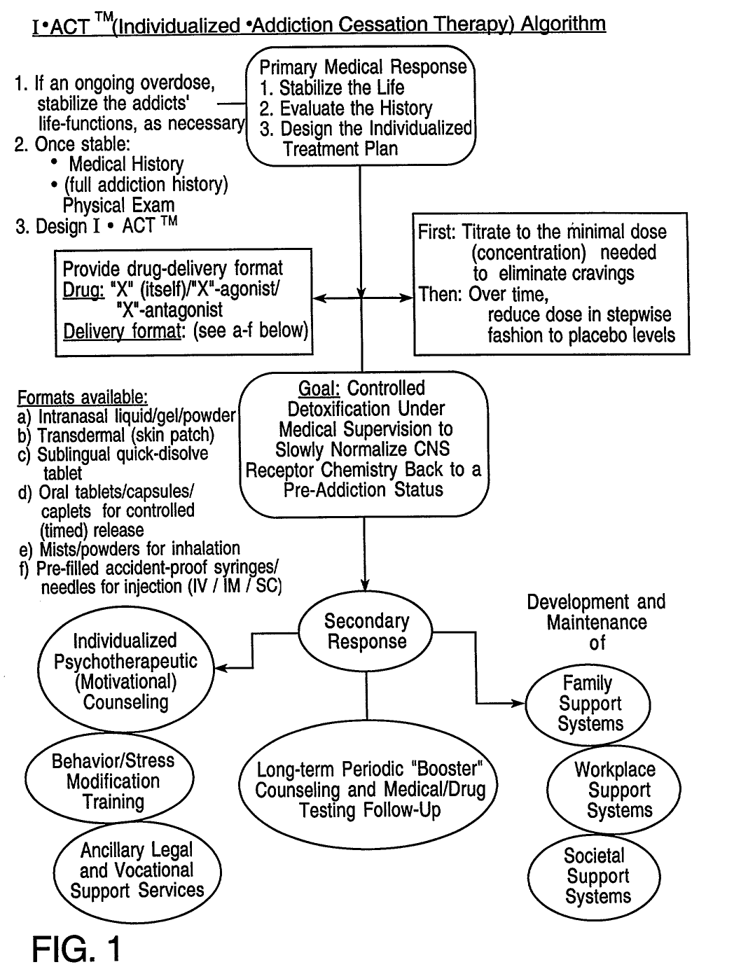

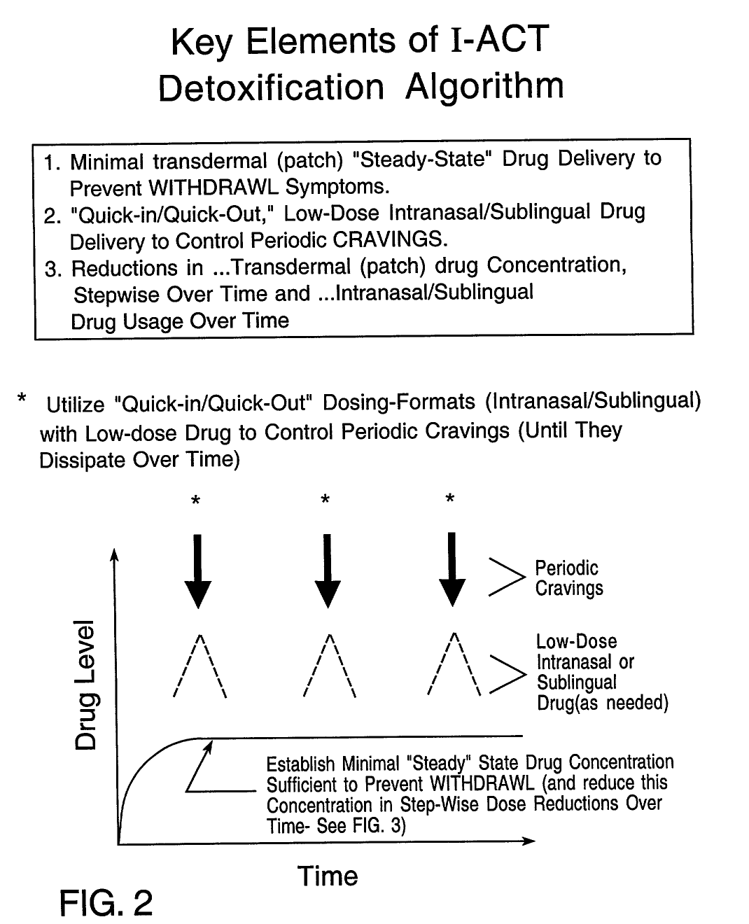

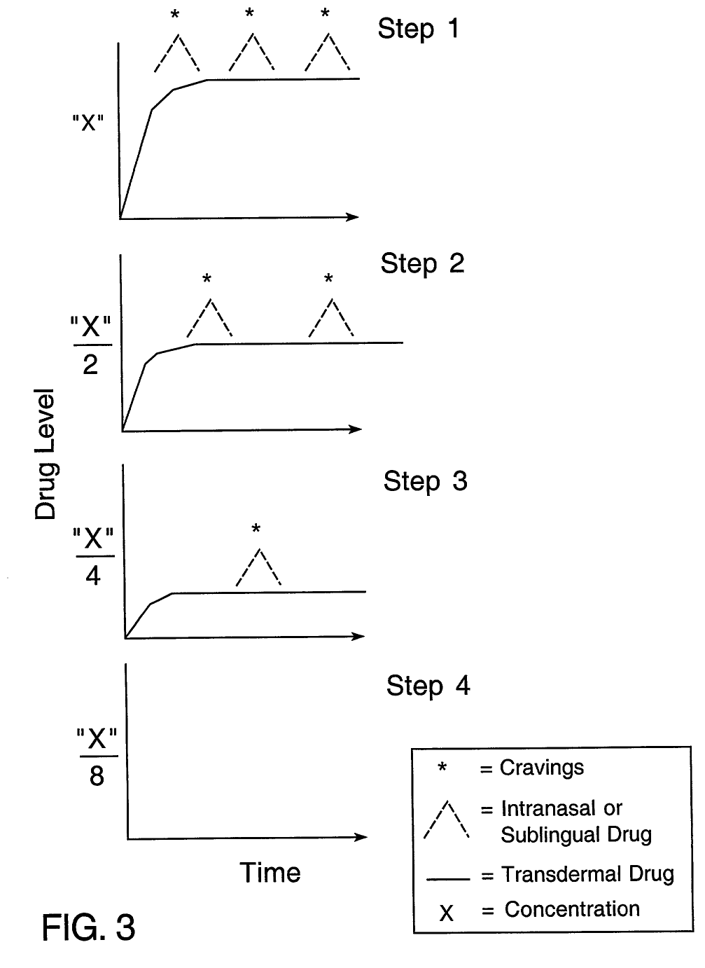

The present invention provides a patient individualized controlled detoxification treatment method for use by a patient dependent upon an addictive drug. This method is set forth in FIGS. 1-3. The method comprises establishing a primary medical response including stabilizing the patient's life functions, obtaining the patient's medical history, and normalizing brain receptor chemistry of the patient to a pre-addictive state over a period of time from about 1 to less than about 365 days, by administering to the patient an individually-titrated, minimal effective dose of the addictive drug, the addictive drug's agonist or the addictive drug's antagonist first via a drug delivery system that establishes a steady state concentration of the addictive drug, the addictive drug's agonist or the addictive drug's antagonist, respectively, which eliminates the patient's addictive drug's withdrawal symptoms. The method further comprises reducing the titrated minimal effective dose of the addictive drug, the addictive drug's agonist or the addictive drug's antagonist, respectively, administered to the patient in a stepwise decreasing fashion over the above-mentioned time period for effecting a decreasing pharmacological concentration to a placebo level of the addictive drug.

In another embodiment of the present invention, a patient individualized controlled detoxification method is provided as described hereinabove and herein, which further includes administering to the patient an effective amount of the addictive drug, the addictive drug's agonist, or the addictive drug's antagonist, respectively, via a second drug delivery system to control the patient's periodic addictive drug cravings.

The treatment methods of the present invention include wherein the first drug delivery system is at least one of the systems selected from the group consisting of a transdermal delivery system, an intranasal delivery system, a sublingual delivery system, an oral delivery system, an inhalation delivery system to the respiratory tract, an intravenous injection delivery system to the blood stream, a subcutaneous injection delivery system, and an intramuscular delivery system.

The treatment methods of the present invention include wherein a second drug delivery system is at least one of the systems selected from the group consisting of an intranasal delivery system to the nasal mucosa, a sublingual delivery system, an intravenous injection delivery system to the blood stream, a subcutaneous injection delivery system, and an intramuscular delivery system.

FIG. 1 shows that the individualized controlled detoxification treatment method of the present invention further comprises establishing for the patient at least one or a combination of secondary responses selected from the group consisting of individualized psychotherapeutic counseling, behavior/stress modification training, ancillary legal and vocational support services, family support systems, workplace support systems, societal support systems, and long-term booster counseling and medical/drug follow-up testing. The individualized controlled detoxification treatment method of the present invention includes wherein the addictive drug is at least one selected from the group consisting of opiods, opiod derivatives, stimulants, depressants, cannabinoids, dissociative anesthetics and hallucinogens. The individualized controlled detoxification treatment method of the present invention includes wherein the addictive drug's agonist is at least one selected from the above-noted group of addictive drugs, and wherein the addictive drug's antagonist is at least one selected from the above-noted group of addictive drugs. Example agonists may include (but are not restricted to) methadone and LAMM, and example antagonists may include (but are not restricted to) naloxene and naltrexone.

The present invention provides pharmaceutical formulations of drugs of addiction (as exemplified by, but not limited to, opioids and morphine derivatives), or their agonists (as exemplified by, but not limited to, methadone/LAMM), or their antagonists (as exemplified by, but not limited to naloxone/naltrexone) that promote detoxification (addiction cessation) in a mammal upon administration to the skin of the mammal via a transdermal delivery system. It is proposed that a dosage unit of a water-soluble buffered gel composition of a pharmaceutically-active amount of drugs such as noted above, with a combination of a neutral pH of about 7.0 and an effective amount of a surfactant, advantageously provides a constant blood plasma concentration following administration of the patch to the skin of the mammal.

The more constant “steady-state” plasma concentration of drugs such as noted above, provides a distinct advantage over the pharmokinetic profile of orally administered drugs in which peak plasma concentrations are not achieved rapidly after administration, and may be variably maintained. For example, it is this “maintenance” level of systemic drug which we propose contributes to the highly addictive nature of methadone/LAMM as it is currently used for detoxification/maintenance therapy.

In accordance with the present invention, one of ordinary skill in the art can adjust the pH of the dosage unit and the amount of the surfactant to provide constant plasma concentrations of drug administration to the skin. Preferably, the pH and the surfactant amount is adjusted to a level that provides a constant plasma concentration following administration of the dosage unit patch to the skin of the mammal.

As a result of providing constant plasma concentrations of drugs following administration to the skin, the transdermal delivery system can advantageously provide reduced peak plasma concentrations as compared to oral dosing. As known in the art, orally administered drugs, as exemplified by the opioid agonists such as methadone/LAMM, reach peak plasma concentration which slowly (and variably) decrease with the passage of time. The dosage units of the present invention exhibit a controlled delivery pattern which in turn facilitates a more constant level in the blood stream of the mammal, thereby minimizing the unwanted side effects (including an enhanced state of addiction) commonly associated with current oral methadone/LAMM therapy.

As previously described, the transdermal dosage units of the present invention will preferably be targeted to have a pH of about 7.0. The pH of the dosage unit (about 7.0) is provided by using a pharmaceutically acceptable buffer system. Examples of buffer systems to be utilized include, but are not limited to, acetate, citrate, carbonate and phosphate buffers.

Pharmaceutically acceptable alkalizers can also be utilized with the buffer system to adjust the pH of the dosage unit, if necessary. Examples of pharmaceutically acceptable alkalizers that can be utilized in conjunction with the buffer system and include, but are not limited to, edetol, potassium carbonate, potassium hydroxide, sodium borate, sodium carbonate, sodium hydroxide and trolamine (triethanolamine).

The surfactant is provided in the amount effective for enhanced delivery of the drug, its agonist, or its antagonist, respectively, to be initiated within minutes of administering the transdermal dosage unit to the skin of the mammal. Stated otherwise, an effective amount of a surfactant is an amount that will allow the dosage unit having a pH of about 7.0 to exhibit pharmaceutically-active plasma concentration of drug within minutes of administration to the skin. The surfactant should be provided in an amount between 0.1 to 1.0 wt. %. However, the exact concentration will be dependent on the pH of the dosage unit, which can be easily ascertained by a skilled artisan.

The surfactant can be any pharmaceutically acceptable surfactant. Examples of suitable surfactants to be utilized include, but are not limited to, salts of long chain hydrocarbons having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetracecyl sulfate. One particularly preferred surfactant is sodium lauryl sulfate (i.e., sodium dodecyl sulfate).

In accordance with the invention, the dosage units contain pharmaceutically-active amounts of drugs of addiction (as exemplified by, but not limited to, methadone/LAMM), or their antagonists (as exemplified by, but not limited to, naloxene/naltrexone). As will be apparent to those skilled in the art, the exact amount required to promote addiction cessation in a mammal will of course depend on the variety of factors. Of chief importance is the extent of addiction, and the drugs(s) chosen to attempt controlled detoxification; of lesser importance is the weight and age of the mammal. In addition, transdermally-delivered drug dosages (due to the improved pharmokinetic profile) can often advantageously provide pharmaceutically-active amounts of plasma drug levels at lower dosages (i.e., less methadone/LAMM can be utilized with the pharmaceutical dosage units of the present invention while providing clinical equivalence to the higher doses, generally required when given orally).

The pharmaceutically-active amounts of the drug of addiction, one of its agonists, or one of its antagonists, respectively, can range widely. In the case of the example opioid agonists, methadone/LAMM, the range of drug in the transdermal system could be for example, but not limited to, between about 1 to 500 milligrams (mg). The actual concentration necessary for a desired effect can easily be ascertained by one of ordinary skill in the art.

The dosage units of the present invention can be provided in any pharmaceutically acceptable form suitable for transdermal delivery to the skin. The dosage units of the present invention can also include other additives such as antioxidants (if required) and preservatives. The amounts utilized will vary with the agents selected and can be easily determined by one of ordinary skill in the art. Pharmaceutically acceptable antioxidants and preservative are employed to increase the shelf life of the composition. The concentration of both the antioxidant (if required) and the preservative will vary with the agents selected.

The present invention also includes a method of detoxification (addiction cessation) in the mammal by administering to the skin of the mammal, the transdermal dosage units described herein.

Another embodiment of the present invention provides pharmaceutical formulations of drugs of addiction (as exemplified by, but not limited to, opioids and morphine derivatives), or their agonists (as exemplified by, but not limited to, methadone/LAMM), or their antagonists (as exemplified by, but not limited to naloxone/naltrexone) that promote detoxification (addiction cessation) in a mammal upon administration to the nasal mucosa of the mammal via unit-dose or multidose delivery systems. It is proposed that a dosage unit of a pharmaceutical vehicle capable of being administered to the nasal mucosa, such as for example but not limited to, aqueous solution or mist, gel or powder, and a pharmaceutically-active effective amount of an addictive drug, its agonist or its antagonist, respectively, incorporated with the pharmaceutical vehicle. One skilled in the art will appreciate that it is preferable that a pH of about 7.0 for the intranasal delivery system is obtained. Further, adding an effective amount of an anionic surfactant via the intranasal delivery system, advantageously provides a peak blood plasma concentration within minutes of administration to the nasal mucosa of the mammal.

The rapid onset of a peak plasma concentration of drugs using the present invention for drugs such as (but not limited to) methadone/LAMM, provides a distinct advantage over the pharmokinetic profile of orally administered methadone/LAMM in which peak plasma concentrations are not achieved rapidly after administration, and may be unnecessarily maintained. It is this “maintenance” level of systemic drug which we propose contributes to the highly addictive nature of methadone/LAMM as it is currently used for detoxification/maintenance therapy.

In accordance with the present invention, one of ordinary skill in the art can adjust the pH of the dosage unit and the amount of the anionic surfactant to provide a peak plasma concentration of drug within minutes of administration to the nasal mucosa. Preferably, the pH and the anionic surfactant amount is adjusted to a level that provides a peak plasma concentration within at least ten minutes of administering the dosage unit to the nasal mucosa of the mammal.

As a result of providing peak plasma concentration within minutes of administration to the nasal mucosa, the dosage units advantageously provide subsequently-reduced drug plasma concentrations once a peak plasma concentration has been achieved, as compared to oral dosing. As known in the art, orally administered drugs, as exemplified by the opioid agonists such as methadone/LAMM, after reaching a peak plasma concentration exhibits a “plateau effect” in which plasma levels slowly decrease with the passage of time. The dosage units of the present invention do not exhibit this plateau effect, which in turn facilitates a more-rapidly reduced level of drug (as exemplified by methadone/LAMM) in the blood stream of the mammal thereby minimizing any unwanted side effects (including an enhanced state of addiction) commonly associated with current oral methadone/LAMM therapy.

As previously described, the intranasal dosage units of the present invention will be targeted to have a pH of about 7.0. The pH of the dosage unit of (about 7.0) is provided by using a pharmaceutically acceptable buffer system. Examples of buffer systems to be utilized include, but are not limited to, acetate, citrate, carbonate and phosphate buffers.

Pharmaceutically acceptable alkalizers can also be utilized with the buffer system to adjust the pH of the dosage unit, if necessary. Examples of pharmaceutically acceptable alkalizers that can be utilized in conjunction with the buffer system include, but are not limited to, edetol, potassium carbonate, potassium hydroxide, sodium borate, sodium carbonate, sodium hydroxide and trolamine (triethanolamine).

The anionic surfactant is provided in the amount effective for a peak plasma concentration of pharmacologically-active formulations (for example methadone/LAMM) to be achieved within minutes of administering the dosage unit to the nasal mucosa of the mammal. Stated otherwise, an effective amount of the anionic surfactant is an amount that will allow the dosage unit having a pH of about 7.0 to exhibit a peak plasma concentration of drug within minutes of administration to the nasal mucosa. The anionic surfactant should be provided in an amount between 0.1 to 1.0 wt. % based upon the total weight percent of the pharmaceutical vehicle and the abusive drug, its agonist, or its antagonist, respectively. However, the exact concentration will be dependent on the pH of the dosage unit, which can be easily ascertained by a skilled artisan.

The anionic surfactant can be any pharmaceutically acceptable anionic surfactant. Examples of suitable anionic surfactants to be utilized include, but are not limited to, salts of long chain hydrocarbons having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetracecyl sulfate. One particularly preferred anionic surfactant is sodium lauryl sulfate (i.e., sodium dodecyl sulfate).

In accordance with the invention, the dosage units contain pharmaceutically-active amounts of drugs of addiction (as exemplified by, but not limited, opioids and morphine-derivatives), or their agonists (as exemplified by, but not limited to, methadone/LAMM), or their antagonists (as exemplified by, but not limited to, naloxone/natrexone).

As will be apparent to those skilled in the art, the exact amount required to promote addiction cessation in a mammal will of course depend on the variety of factors. Of chief importance is the extent of addiction, and the drug(s) chosen to attempt controlled detoxification; of lesser importance is the weight and age of the mammal. In addition, intranasal dosages of (for example) methadone/LAMM (due to the improved pharmokinetic profile) can advantageously provide pharmaceutically-active amounts of methadone/LAMM plasma levels at lower dosages (i.e., less methadone/LAMM can be utilized with the pharmaceutical dosage units of the invention while providing clinical equivalence to the higher doses generally required when given orally).

The pharmaceutically-active amounts of the drug of addiction, one of its agonists (for example, methadone/LAMM), or one of its antagonists of the drug delivery systems of the present invention can range widely, such as for example but not limited to, about 1 to 500 milligrams, and such as for example, but not limited to 0.2 to 20 milligrams (mg) per dose in the case of methadone/LAMM.

The dosage units of the present intranasal drug delivery system invention can range from, for example but not limited to, 0.1 to 0.4 ml. (milliliter) per dose. The actual concentration necessary for a desired effect can easily be ascertained by one of ordinary skill in the art. The dosage units of the present invention can be provided in any pharmaceutically acceptable form suitable for administration to the nasal mucosa.

In another embodiment of the present invention, the pharmaceutical formulations can be dehydrated to form a powder dosage unit, which can be administered to the nasal mucosa. The powder dosage units can be administered neat, or in conjunction with a pharmaceutically acceptable carrier. In a preferred embodiment, the powder formulation is incorporated into a microparticulate often referred to as microspheres or nanospheres. Processes for incorporating pharmaceuticals into such microparticulates are well known in the art.

The dosage units of the present invention can also include other additives such as for example, but not limited to, humectants and preservatives. A humectant or soothening agent is utilized to inhibit drying of the nasal mucosa and to prevent irritation. Any pharmaceutical acceptable humectant can be utilized, in which examples include, but are not limited to, sorbitol, propylene glycol and glycerol. The amounts utilized will vary with the agent selected and can be easily determined by one of ordinary skill in the art.

A pharmaceutically acceptable preservative may be employed to increase the shelf life of the intranasal drug delivery system. Any pharmaceutically acceptable preservative can be utilized, such as for example, including, but not limited to, thimerosal, chlorobutanol, benzyl alcohol, parabens, and benzalkonium chloride. Preferably, benzalkonium chloride is utilized. The concentration of the preservative will range from 0.2 to 2 wt. %, although the actual concentration will vary with the preservative selected.

The dosage units may also be isotonic, although isotonicity is not required. Typically, pharmaceutically acceptable agents such as dextrose, boric acid, sodium tartarate, propylene glycol and other inorganic or organic solutes can be utilized to adjust tonicity. Sodium chloride is particularly preferred if a buffer system containing sodium is utilized.

The present invention also includes a method of detoxification (addiction cessation) in the mammal comprising administration to the nasal mucosa of the mammal the intranasal dosage units described herein.

In another embodiment of the present invention, pharmaceutical formulations of drugs of addiction, (as exemplified by, but not limited to, opioids and morphine derivatives), or their agonists (as exemplified by, but not limited to, methadone/LAMM), or their antagonists (as exemplified by, but not limited to, naloxone/naltrexone), are provided for by promoting detoxification (addiction cessation) in a mammal upon administration to the oral mucosa below the tongue of the mammal via fast-dissolve unit-dose sublingual delivery systems. More preferably, a dosage unit of a pharmaceutically-active amount of drugs such as methadone/LAMM with a combination of a pH of 7.0 and an effective amount of anionic surfactant, advantageously provides a pharmaceutically-active blood plasma concentration within minutes of administration to the oral mucosa below the tongue of the mammal.

The relatively rapid onset of a peak plasma concentration of fast-dissolve sublingual drugs as exemplified by (but not limited to) methadone/LAMM provides a distinct advantage over the pharmokinetic profile of orally administered methadone/LAMM in which peak plasma concentrations are not achieved rapidly after administration, and may be unnecessarily maintained at higher than necessary doses. It is this high “maintenance” level of systemic drug which we propose contributes to the highly addictive nature of methadone/LAMM as it is currently used for detoxification/maintenance therapy.

In accordance with the present invention, one of ordinary skill in the art can adjust the pH of the dosage unit and the amount of the anionic surfactant to provide a peak plasma concentration of drug within minutes of administration to the oral mucosa below the tongue. Preferably, the pH and the anionic surfactant amount is adjusted to a level that provides a peak plasma concentration within at least ten minutes of administering the dosage unit to the sublingual mucosa of the mammal.

As a result of providing peak plasma concentrations within minutes of administration to the sublingual mucosa, the dosage units of the sublingual delivery system of the present invention advantageously provide subsequently reduced drug plasma concentrations once a peak plasma concentration has been achieved, as compared to current oral dosing procedures. As will be understood by those person skilled in the art, orally administered drugs, as exemplified by the opioid agonists such as methadone/LAMM, after reaching a peak plasma concentration exhibits a “plateau effect” in which plasma levels slowly decrease with the passage of time. The dosage units of the present invention are targeted not to exhibit such a plateau effect, which in turn facilitates a more-rapidly reduced level of drug (as exemplified by methadone/LAMM) in the blood stream of the mammal thereby minimizing any unwanted side effects (including an enhanced state of addiction) commonly associated with current oral methadone/LAMM therapy.

The sublingual dosage units of the present invention may preferably have a pH of about 7.0. The pH of the dosage unit (about 7.0) is provided by using a pharmaceutically acceptable buffer system. Examples of buffer systems to be utilized include, but are not limited to, acetate, citrate, carbonate and phosphate buffers. The sublingual drug delivery system of the present invention preferably has a formulation that begins dissolution upon the mammal's sublingual mucosa in about 0.01 to 600 seconds of time.

Pharmaceutically acceptable alkalizers can also be utilized with the buffer system to adjust the pH of the dosage unit, if necessary. Examples of pharmaceutically acceptable alkalizers that can be utilized in conjunction with the buffer system include, but are not limited to, edetol, potassium carbonate, potassium hydroxide, sodium borate, sodium carbonate, sodium hydroxide and trolamine (triethanolamine).

The anionic surfactant is provided in the amount effective for a peak plasma concentration of (for example) methadone/LAMM to be achieved within minutes of administering the dosage unit to the sublingual mucosa of the mammal. Stated otherwise, an effective amount of the anionic surfactant is an amount that will allow the dosage unit having a pH of about 7.0 to exhibit a peak plasma concentration of drug within minutes of administration to the sublingual mucosa. The anionic surfactant should be provided in an amount between 0.1 and 1.0 wt. %. However, the exact concentration will be dependent on the pH of the dosage unit, which can be easily ascertained by a skilled artisan.

The anionic surfactant can be any pharmaceutically acceptable anionic surfactant. Examples of suitable anionic surfactants to be utilized include, but are not limited to, salts of long chain hydrocarbons having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetracecyl sulfate. A preferred anionic surfactant is sodium lauryl sulfate (i.e., sodium dodecyl sulfate).

In accordance with the invention, the dosage units contain pharmaceutically-active amounts of drugs of addiction (as exemplified by, but not limited to, opioids and morphine-derivatives), or their agonists (as exemplified by, but not limited to, methadone/LAMM), or their antagonists (as exemplified by, but not limited to, naloxene/naltrexone).

As will be appreciated by those persons skilled in the art, the exact amount required to promote addiction cessation in a mammal will of course depend on the variety of factors. Of chief importance is the extent of addiction, and the drug(s) chosen to attempt controlled detoxification; of lesser importance is the weight and age of the mammal. In addition, intranasal dosages of methadone/LAMM (due to the improved pharmokinetic profile) can advantageously provide pharmaceutically-active amounts of methadone/LAMM plasma levels at lower dosages. (i.e. less methadone/LAMM can be utilized with the pharmaceutical dosage units of the invention while providing clinical equivalence to the higher doses generally required when given orally).

The dosage units of the present invention can be provided in any pharmaceutically acceptable form suitable for administration to the sublingual mucosa.

In another embodiment of the present invention, the pharmaceutical formulations can be dehydrated to form a powder dosage unit, which can be administered to the sublingual mucosa, or in conjunction with a pharmaceutically acceptable carrier, preferably incorporated into a microparticulate often referred to as microspheres or nanospheres. Processes for incorporating pharmaceuticals into such microparticulates are well known by those person in the art.

The dosage units of the present invention may also include other additives such as antioxidants (if preferable) and preservatives. Any pharmaceutically acceptable antioxidant can be utilized; the amount utilized will vary with the agent selected and can be easily determined by one of ordinary skill in the art.

In another embodiment of this invention, a pharmaceutically acceptable preservative is also employed to increase the shelf life of the sublingual delivery system of the present invention. Any pharmaceutically acceptable preservative can be utilized with examples, including, but not limited to, thimerosal, chlorobutanol, benzyl alcohol, parabens, and benzalkonium chloride. Preferably, benzalkonium chloride is utilized. The concentration of the preservative will range from 0.2 to 2 wt. %, although the actual concentration will vary with the preservative selected.

The dosage units of the present invention may also be isotonic, although isotonicity is not required. Typically, pharmaceutically acceptable agents such as dextrose, boric acid, sodium tartarate, propylene glycol and other inorganic or organic solutes can be utilized to adjust tonicity. Sodium chloride is particularly preferred if a buffer system containing sodium is utilized.

The present invention also includes a method of detoxification (addiction cessation) in the mammal comprising administration to the sublingual mucosa of the mammal the sublingual dosage units described herein.

Another embodiment of the present inventions provides a computer database system having a collection of information from patients through various sources, including but not limited to, questionnaires, patient interviews, medical history and determinations, and clinical results from the dispersion of the employment of the individualized addiction cessation formulations and methods of the present invention. The data that is collected will be input into a centralized database system and using a mathematical and statistical analysis process, the system assists providers in making information and qualitative decisions with regard to administering the proper medical treatments. The database system accepts input from remote sources in real time. Analysis and reports are then created from the sources using statistical processing tables and comparing data in the progressively growing and maturing database of information. All information is held in a tightly controlled security environment as well as being encrypted so that patients' identity is not revealed. Information will be collected in a timely manner on various forms designed to work in conjunction with the various phases of the individualized addiction cessation therapy so as to determine a trend. This, in turn, will help determine when patients are ready to progress onto various levels of the treatment. In addition, psychotherapists perform evaluations of the patients as well as laymen in the field in order to develop a broad perspective of the patient's condition. The database engines look for key phrases and word “logy” to help recognize critical points in the therapy. This will both assist with the advancement of the patient and in the recognition of changes in treatment patterns.

The method of the present invention for developing a treatment plan for a new patient having a substance addiction for purposes of administering various phases of cessation treatment comprises collecting information from other patients as treatment is administered, storing the collected information in a database, developing trends from other patients' treatments based upon the collected information, and analyzing the trends and applying them to the new patient for purposes of establishing a treatment protocol relative to the new patient. The collected information is categorized in such a way that it is dependent upon the current phase of treatment for the patient. This method further includes predicting the medical outcome of the new patient wherein the collected information includes information regarding both treatment and medical outcome. The method also includes a further step of recognizing, based on the trend, when a patient has progressed to a new phase, and when the treatment for the new patient should be modified accordingly.

It will be appreciated by those skilled in the art that up to this date, there has not been this type of method for developing a treatment plan for addicted patients and to provide a clearinghouse of information. It will be understood by those persons skilled in the art that the computer database system and methods of the present invention shall build and strengthen support groups as well as professional medical practitioners. In addition, it will assist drug manufacturers in having a broad clinical track record for their pharmaceuticals that are administered to the addicted patients.

Whereas, particular embodiments of the present invention have been described herein for the purpose of illustration. It will be evident to those skilled in the art that numerous variations of the details of the present invention may be made without departing from the invention as defined in the appended claims.

We claim:

1. A patient individualized controlled detoxification treatment method for use by a patient dependent upon an addictive drug comprising:
establishing a primary medical response including stabilizing the patient's life functions, obtaining the patient's medical history, and normalizing brain receptor chemistry of the patient to a pre-addictive state over a period of time from about 1 to less than 365 days, by administering to the patient an individually-titrated minimal effective dose of the addictive drug, the addictive drug's agonist or the addictive drug's antagonist, via a first drug delivery system that establishes a steady state concentration of said addictive drug, the addictive drug's agonist or the addictive drug's antagonist, respectively, which eliminates the patient's addictive drug's withdrawal symptoms, and then
reducing said titrated minimal effective dose of said addictive drug, the addictive drug's agonist or the addictive drug's antagonist, respectively, administered to the patient in a stepwise decreasing fashion over said time period for effecting a decreasing pharmacological concentration to a placebo level of the addictive drug.

2. The individualized controlled detoxification treatment method of claim 1 further comprising administering to the patient an effective amount of the addictive drug, the addictive drug's agonist, or the addictive drug's antagonist, respectively, via a second drug delivery system to control the patient's periodic addictive drug cravings.

3. The individualized controlled detoxification treatment method of claim 1 including wherein said first drug delivery system is at least one of the systems selected from the group consisting of a transdermal delivery system, an intranasal delivery system, a sublingual delivery system, an oral delivery system, an inhalation delivery system to the respiratory tract, an intravenous injection delivery system to the blood stream, a subcutaneous injection delivery system, and an intramuscular delivery system.

4. The individualized controlled detoxification treatment method of claim 2 including wherein said second drug delivery system is at least one of the systems selected from the group consisting of an intranasal delivery system, a sublingual delivery system, an intravenous injection delivery system to the blood stream, a subcutaneous injection delivery system, and an intramuscular delivery system.

5. The individualized controlled detoxification treatment method of claim 1 further comprising establishing for the patient at least one or a combination of secondary responses selected from the group consisting of individualized psychotherapeutic counseling, behavior/stress modification training, ancillary legal and vocational support services, family support systems, workplace support systems, societal support systems, and long-term booster counseling and medical/drug follow-up testing.

6. The individualized controlled detoxification treatment method of claim 1, including wherein said addictive drug is at least one selected from the group consisting of opiods, opiod derivatives, stimulants, depressants, cannabinoids, dissociative anesthetics and hallucinogens.

7. The individualized controlled detoxification treatment method of claim 1 including wherein said addictive drug's agonist is at least one selected from the group consisting of methadone and levomethadyl acetate.

8. The individualized controlled detoxification treatment method of claim 1 including wherein said addictive drug's antagonist is at least one selected from the group consisting of naloxone and naltrexone.

9. A transdermal drug delivery system for promoting detoxification of a mammal dependent upon an addictive drug comprising:
a transdermal pharmaceutical vehicle, and
a pharmaceutically active effective amount of an addictive drug, the addictive drug's agonist, or the addictive drug's antagonist contained within said pharmaceutical vehicle and capable of being released from said transdermal pharmaceutical vehicle over time to prevent drug withdrawal symptoms from occurring in the mammal.

10. The transdermal drug delivery system of claim 9 further comprising an effective amount of at least one of the group consisting of a surfactant, an antioxidant, and a preservative, and combinations thereof.

11. The transdermal drug delivery system of claim 10 wherein said surfactant is a salt of a long chain hydrocarbon with a functional group selected from the group consisting of carboxylates, sulfonates and mixtures thereof, a salt of a long chain hydrocarbon with a sulfate functional group.

12. The transdermal drug delivery system of claim 11 wherein said surfactant is sodium lauryl sulfate.

13. The transdermal drug delivery system of claim 9 capable of maintaining in the mammal a constant blood plasma concentration of said addictive drug, said addictive drug's agonist, or said addictive drug's antagonist after administering the transdermal drug delivery system to the skin.

14. The transdermal drug delivery system of claim 9 wherein the amount of addictive drug, addictive drug agonist or addictive drug antagonist is from about 1.0 to 500.0 milligrams.

15. The transdermal drug delivery system of claim 10 having from about 0.1 to 1.0 weight percent of said surfactant.

16. A method of detoxifying a mammal that is dependent upon an addictive drug comprising:
administering to the skin of the mammal a dosage unit comprising a transdermal pharmaceutical vehicle and a pharmaceutically active effective amount of an addictive drug, the addictive drug's agonist, or the addictive drug's antagonist contained within said transdermal pharmaceutical vehicle and capable of being released from said transdermal pharmaceutical vehicle over time, to prevent drug withdrawal symptoms from occurring in the mammal.

17. An intranasal drug delivery system for promoting detoxification of a mammal dependent upon an addictive drug comprising:
a pharmaceutical vehicle capable of being administered to the nasal mucosa, and
a pharmaceutically active effective amount of an addictive drug, the addictive drug's agonist, or the addictive drug's antagonist incorporated with said pharmaceutical vehicle.

18. The intranasal drug delivery system of claim 17, wherein said system has a pH of about 7.0.

19. The intranasal drug delivery system of claim 17 further comprising an effective amount of at least one of the group consisting of a surfactant, an antioxidant, and a preservative, and combinations thereof.

20. The intranasal drug delivery system of claim 19 wherein said surfactant is a salt of a long chain hydrocarbon with a functional group selected from the group consisting of carboxylates, sulfonates and mixtures thereof or a salt of a long chain hydrocarbon with a sulfate functional group.

21. The intranasal drug delivery system of claim 20 wherein said surfactant is sodium lauryl sulfate.

22. The intranasal drug delivery system of claim 17 capable of maintaining in the mammal a pharmaceutically-active blood plasma concentration of said addictive drug, said addictive drug's agonist, or said addictive drug's antagonist after administering the intranasal drug delivery system to the nasal mucosa of the mammal.

23. The intranasal drug delivery system of claim 17 wherein the amount of addictive drug, addictive drug agonist, or addictive drug antagonist is from about 1.0 to 500.0 milligrams.

24. The intranasal drug delivery system of claim 17 having from about 0.1 to 1.0 weight percent of said surfactant.

25. A method of detoxifying a mammal that is dependent upon an addictive drug comprising: administering to the nasal mucosa of the mammal a dosage unit comprising a pharmaceutical vehicle capable of being administered to the nasal mucosa and a pharmaceutically active effective amount of an addictive drug, the addictive drug's agonist or the addictive drug's antagonist incorporated with said pharmaceutical vehicle to prevent drug withdrawal symptoms from occurring in the mammal.

26. The intranasal drug delivery system of claim 17 wherein said pharmaceutical vehicle is selected from the group consisting of an aqueous buffered solution, a gel, and a powder.

27. The intranasal drug delivery system of claim 19 wherein said surfactant is an anionic surfactant.

28. A sublingual drug delivery system for promoting detoxification of a mammal dependent upon an addictive drug comprising:
a pharmaceutical vehicle capable of being administered to effect dissolution upon the mammal's sublingual mucosa, and
a pharmaceutically active effective amount of an addictive drug, the addictive drug's agonist, the addictive drug's antagonist incorporated with said pharmaceutical vehicle.

29. The sublingual drug delivery system of claim 28 wherein said system has a pH of about 7.0.

30. The sublingual drug delivery system of claim 28 further comprising an effective amount of at least one of the group consisting of a surfactant, an antioxidant, and a preservative, and combinations thereof.

31. The sublingual drug delivery system of claim 30 wherein said surfactant is a salt of a long chain hydrocarbon with a functional group selected from the group consisting of carboxylates, sulfonates and mixtures thereof, or a salt of a long chain hydrocarbon with sulfate functional group.

32. The sublingual drug delivery system of claim 31 wherein said surfactant is sodium lauryl sulfate.

33. The sublingual drug delivery system of claim 28 capable of maintaining in the mammal a pharmaceutically-active blood plasma concentration of said addictive drug, said addictive drug's agonist, or said addictive drug's antagonist after administering the sublingual drug delivery system to the mammal's sublingual mucosa.

34. The sublingual drug delivery system of claim 28 wherein the amount of addictive drug, addictive drug agonist, or addictive drug antagonist is from about 1.0 to 500.0 milligrams.

35. The sublingual drug delivery system of claim 28 having from about 0.1 to 1.0 weight percent of said surfactant.

36. A method of detoxifying a mammal that is dependent upon an addictive drug comprising:
administering under the tongue of the mammal a dosage unit comprising a pharmaceutical vehicle capable of being administered to effect dissolution upon the mammal's sublingual mucosa and a pharmaceutically active effective amount of an addictive drug, the addictive drug's agonist, or the addictive drug's antagonist incorporated with said pharmaceutical vehicle to prevent drug withdrawal symptoms from occurring in the mammal.

37. The sublingual drug delivery system of claim 28 wherein said pharmaceutical vehicle is an aqueous buffered formulation that begins dissolution upon the mammal's sublingual mucosa in about 0.01 to 600.0 seconds of time.

38. The sublingual drug delivery system of claim 30, wherein said surfactant is an anionic surfactant.

39. A method for developing a treatment plan for a new patient for purposes of administering various phases of treatment to the new patient comprising the steps of:
collecting information from other patients as treatment is administered;
storing said collected information in a database;
developing trends from other patients' treatments, based upon said collected information; and
analyzing said trends and applying them to said new patient for purposes of establishing a treatment protocol relative to said new patient.

40. The method of claim 39 including wherein said collected information includes information regarding both treatment and medical outcome.

41. The method of claim 39 including predicting the medical outcome of said new patient.

42. The method of claim 40 including wherein said collected information is dependent upon the current phase of treatment for said patient.

43. The method of claim 39 further comprising the step of recognizing, based upon said trends, when a patient has progressed to a new phase and when said treatment for said patient should be modified.
